(19) United States
(12) Patent Application Publication
(10) Pub. No.: US 2007/0099195 A1
(43) Pub. Date: May 3, 2007

(54) Methods and compositions for separating nucleic acids from a solid support

(76) Inventors: Xiaohua C. Huang, Mountain View, CA (US); Eric M. LeProust, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/266,049

(22) Filed: Nov. 2, 2005

(51) Int. Cl. C12Q 1/68; C07H 21/04

(52) U.S. Cl. 435/6; 536/25.4

(57) ABSTRACT

Methods and compositions for separating nucleic acids from a solid support are provided. In the subject methods, a silica containing solid support having nucleic acids immobilized on a surface thereof is subjected to cleavage conditions, such that a fluid cleavage product that includes nucleic acids and silica is produced. The resultant fluid cleavage product is then purified to produce a final nucleic acid composition that includes a substantially reduced amount of silica. Also provided are kits for practicing the subject methods.

### INTRODUCTION

Chemical arrays, such as nucleic acid and protein arrays, are finding increasing use in a variety of different applications, and in doing so are making a significant impact in a variety of different fields, including research, medicine, and the like. In many instances, arrays include regions of usually different composition arranged in a predetermined configuration on a substrate. These regions (sometimes referenced as “features”) are positioned at known respective locations (“addresses”) on the substrate and are therefore “addressable.”

Arrays can be fabricated by depositing previously obtained biopolymers onto a substrate, or by in situ synthesis methods. The in situ fabrication methods include those described in U.S. Pat. Nos. 5,449,754 and 6,180,351 as well as published PCT application no. WO 98/41531 and the references cited therein. Further details of fabricating biopolymer arrays are described in U.S. Pat. Nos. 6,242,266; 6,232,072; 6,180,351 and U.S. Pat. No. 6,171,797. Other techniques for fabricating biopolymer arrays include known light directed synthesis techniques.

Recently chemical arrays have been employed for the rapid production of a defined mixture of nucleic acids, such as oligonucleotides, that may be synthesized directly on the surface of a substrate. Once synthesized, however, the nucleic acids need to be separated from the surface of the substrate. One method for separating nucleic acids from the surface of the substrate uses a cleavable linker that attaches the nucleic acids to the substrate. However, this approach is not entirely satisfactory, as it requires that a built in cleavable linker be included into the surface immobilized nucleic acid which must be cleaved so as to remove the nucleic acid from the substrate.

A method for separating the nucleic acid from the surface of the substrate that does not require the use of a cleavable linker would be beneficial. As such, there is continued interest in the identification of additional methods of using nucleic acid arrays as a source of nucleic acids. The present technology provides such a method.

### SUMMARY OF THE INVENTION

Methods and compositions for separating nucleic acids from a solid support (e.g., an array) are provided. In representative embodiments, a silica containing solid support having nucleic acids immobilized on a surface thereof is subjected to cleavage conditions such that a fluid cleavage product which includes nucleic acids and silica is produced. The resultant fluid cleavage product is then purified to produce a final nucleic acid composition that includes a substantially reduced amount of silica, as compared to the fluid cleavage product. The resultant final nucleic acid composition includes solution phase nucleic acids that may then be used in a variety of applications. Also provided are kits for practicing the subject methods.

Aspects of the invention involve methods of separating a nucleic acid from a surface of a solid support that includes silica (e.g., glass), to which the nucleic acid is immobilized, e.g., bonded. The subject methods include: (a) contacting the surface of the solid support with a cleavage agent, such as a base (e.g., ammonia), that is sufficient to separate the nucleic acid from the surface of the solid support and thereby produce a fluid cleavage product that includes both the separated nucleic acid and the silica; and (b) purifying the fluid cleavage product to obtain a final composition that includes the nucleic acids, where the amount of silica present that is substantially reduced as compared to the amount of silica present in the fluid cleavage product.

In certain embodiments, the contacting of the surface of the solid support with the cleavage agent is for an amount of time sufficient to separate the nucleic acid from the surface of the solid support. In other certain embodiments, purifying the fluid cleavage product includes a physical separation step, such as a physical separation based on density, for instance: centrifugation, filtration, extraction, distillation, and/or decanting. In further embodiments, purifying the fluid cleavage product includes one or more precipitation steps, for instance with an alcohol, such as ethanol, isopropanol, or the like. In additional embodiments, purifying the fluid cleavage product may include both a physical separation step and one or more precipitation steps.

In certain embodiments, the nucleic acids are bonded to the substrate in such a way as to form an array of nucleic acids. The array of nucleic acids may include at least about 100 different features of nucleic acids and may be at a density of at least about 10 features/cm². Thus, in particular embodiments, the purification step results in a reduction of silica in the final nucleic acid composition by about 75%, wherein the final amount of nucleic acids in the composition is about 40%, as compared to the initial amounts of silica and nucleic acids in the fluid cleavage product.

In certain embodiments, the end result of the purification step will be a composition of nucleic acids that contains a reduced amount of silica. The final composition of nucleic acids may include any type of nucleic acid, such as ribose nucleic acids (RNA), deoxyribose nucleic acids (DNA), nucleic acids having made from nucleotide analogs, etc. In representative embodiments, the nucleic acids may be anywhere from about 10 to about 250 nucleotides in length, such as from about 15 to about 150 nucleotides in length, including from about 20 to about 100 nucleotides in length, e.g., about 40 to about 80 nucleotides in length.

Also provided are kits that include: (a) a cleavage agent for separating nucleic acids from a silica comprising support; and (b) a purification element. In certain embodiments, the kits further include a silica comprising support to which a nucleic acid is bonded, as reviewed above.

### BRIEF DESCRIPTION OF THE FIGURES

FIG. 1A shows a graph of an optical density (OD) reading of a sample containing silica and nucleic acids prior to ethanol precipitation, as reported in the experimental section, below.

FIG. 1B shows a graph of an OD reading of the sample after a first ethanol precipitation, as reported in the experimental section, below.

FIG. 1C shows a graph of an OD reading of the sample after a second ethanol precipitation as reported in the experimental section, below.

### DEFINITIONS

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes DNA (including cDNA), RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.

An “oligonucleotide” generally refers to a nucleotide multimer of about 10 to 100 nucleotides in length, while a “polynucleotide” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region. For example, each region may extend into a third dimension in the case where the substrate is porous while not having any substantial third dimension measurement (thickness) in the case where the substrate is non-porous. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable set of probes” and analogous terms refers to the multiple regions of different moieties supported by or intended to be supported by the array surface.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

“Hybridizing” and “binding”, with respect to polynucleotides, are used interchangeably.

A “plastic” is any synthetic organic polymer of high molecular weight (for example at least 1,000 grams/mole, or even at least 10,000 or 100,000 grams/mole.

“Flexible” with reference to a substrate or substrate web (including a housing or one or more housing component such as a housing base and/or cover), references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C. “Rigid” refers to a substrate (including a housing or one or more housing component such as a housing base and/or cover) which is not flexible, and is constructed such that a segment about 2.5 by 7.5 cm retains its shape and cannot be bent along any direction more than 60 degrees (and often not more than 40, 20, 10, or 5 degrees) without breaking.

When one item is indicated as being “remote” from another, this descriptor indicates that the two items are at least in different buildings, and may be at least one mile, ten miles, or at least one hundred miles apart. When different items are indicated as being “local” to each other they are not remote from one another (for example, they can be in the same building or the same room of a building). “Communicating”, “transmitting” and the like, of information reference conveying data representing information as electrical or optical signals over a suitable communication channel (for example, a private or public network, wired, optical fiber, wireless radio or satellite, or otherwise). Any communication or transmission can be between devices which are local or remote from one another. “Forwarding” an item refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless). “Receiving” something means it is obtained by any possible means, such as delivery of a physical item (for example, an array or array carrying package). When information is received it may be obtained as data as a result of a transmission (such as by electrical or optical signals over any communication channel of a type mentioned herein), or it may be obtained as electrical or optical signals from reading some other medium (such as a magnetic, optical, or solid state storage device) carrying the information. However, when information is received from a communication it is received as a result of a transmission of that information from elsewhere (local or remote).

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. Items of data are “linked” to one another in a memory when a same data input (for example, filename or directory name or search term) retrieves those items (in a same file or not) or an input of one or more of the linked items retrieves one or more of the others. In particular, when an array layout is “linked” with an identifier for that array, then an input of the identifier into a processor which accesses a memory carrying the linked array layout retrieves the array layout for that array.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination which will perform those or equivalent steps. Programming may be accomplished, for example, from a computer readable medium carrying necessary program code (such as a portable storage medium) or by communication from a remote location (such as through a communication channel).

A “memory” or “memory unit” refers to any device which can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “assembly” includes a substrate and at least one chemical array on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Reading” signal data from an array refers to the detection of the signal data (such as by a detector) from the array. This data may be saved in a memory (whether for relatively short or longer terms).

A “package” is one or more items (such as an array assembly optionally with other items) all held together (such as by a common wrapping or protective cover or binding). Normally the common wrapping will also be a protective cover (such as a common wrapping or box) which will provide additional protection to items contained in the package from exposure to the external environment. In the case of just a single array assembly a package may be that array assembly with some protective covering over the array assembly (which protective cover may or may not be an additional part of the array unit itself).

It will also be appreciated that throughout the present application, that words such as “cover”, “base” “front”, “back”, “top”, “upper”, and “lower” are used in a relative sense only.

“May” refers to optionally.

When two or more items (for example, elements or processes) are referenced by an alternative “or”, this indicates that either could be present separately or any combination of them could be present together except where the presence of one necessarily excludes the other or others.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions that determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2 SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

As such, the term “hybridization” refers to the formation of a duplex structure by two single stranded nucleic acids due to complementary base pairing. Hybridization can occur between exactly complementary nucleic acid strands or between nucleic acid strands that contain minor regions of mismatch. As used herein, the term “substantially-complementary” refers to sequences that are complementary except for minor regions of mismatch, wherein the total number of mismatched nucleotides is no more than about 3 for a sequence about 15 to about 35 nucleotides in length. Conditions under which only exactly complementary nucleic acid strands will hybridize are referred to as “stringent” or “sequence-specific” hybridization conditions. Stable duplexes of substantially complementary nucleic acids can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs. Computer software for calculating duplex stability is commercially available from a variety of vendors.

Stringent, sequence-specific hybridization conditions, under which an oligonucleotide will hybridize only to the exactly complementary target sequence, are well known in the art (see, e.g., Sambrook et al., 2001, Molecular Cloning—A Laboratory Manual, Cold Spring Harbor Laboratory, Cold Spring Harbor, N.Y., incorporated herein by reference). Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the base pairs have dissociated. Relaxing the stringency of the hybridizing conditions allows sequence mismatches to be tolerated; the degree of mismatch tolerated can be controlled by suitable adjustment of the hybridization conditions.

### DETAILED DESCRIPTION OF REPRESENTATIVE EMBODIMENTS

Methods and compositions for separating nucleic acids from a solid support (e.g., an array) are provided. In embodiments of the subject methods, a silica containing solid support having nucleic acids immobilized on a surface thereof is subjected to cleavage conditions, such that a fluid cleavage product that includes nucleic acids and silica is produced. The resultant fluid cleavage product is then purified to produce a nucleic acid composition that includes a substantially reduced amount of silica. The resultant nucleic acid composition comprises solution phase nucleic acids that may then be used in a wide variety of applications. Also provided are kits for practicing the subject methods.

Before the present invention is further described, it is to be understood that this invention is not limited to the particular embodiments described herein, as such may, of course, vary. It is also to be understood that the terminology used herein is for the purpose of describing particular representative embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. The figures shown herein are not necessarily drawn to scale, with some components and features being exaggerated for clarity.

As summarized above, aspects of the invention include first generating an initial fluid cleavage product from a silica containing support having nucleic acids bonded to a surface thereof, wherein the initial fluid cleavage product contains both solution phase nucleic acids and silica. The initial fluid cleavage product is then purified to separate the nucleic acids from the silica. The resultant purified nucleic acids may then be used for a variety of applications, as will be described in more detail below.

The silica containing support having nucleic acids immobilized on a surface thereof that is employed in the subject methods is, in representative embodiments, a silica containing substrate having a planar surface on which is immobilized one or more distinct features of nucleic acids, e.g., as is found in a nucleic acid array. Embodiments of the subject methods that employ a nucleic acid array as the starting source of nucleic acids may be viewed as array-based methods. In certain embodiments where a nucleic acid array is employed as the source of nucleic acids, the array is an in situ nucleic acid array.

Where in situ nucleic acid arrays are employed as starting materials in the subject methods, the in situ fabricated nucleic acid arrays can be fabricated using any convenient protocol. In representative embodiments, such arrays may be produced by depositing multiple different reagent droplets by pulse jet or other means at a given target location on a substrate surface in order to produce a nucleic acid feature. Representative in situ fabrication methods include those described in U.S. Pat. Nos. 6,180,351; 6,919,181; and published U.S. Application US 2004-0018498 A1.

The sequences of the nucleic acids of the various features of the array may vary greatly depending on the intended use of the product nucleic acids to be produced using the subject methods. For example, in applications where the product nucleic acids are used as template nucleic acids for the generation of yet additional nucleic acids, the sequences of the nucleic acids will be chosen in view of the desired templates. In other representative embodiments where the methods are employed to produce libraries of modulating nucleic acids, such as shRNA molecules, decoy molecules, antisense molecules, etc., the sequences of the nucleic acids on the initial array are selected in view of the genes whose expression is desired to be modulated with the final product composition. In certain embodiments, the methods include designing the sequences of the nucleic acids that are present array. In such embodiments, the sequences may be selected based on the desired use of the purified nucleic acids that are to be produced. For example, where the purified nucleic acids are oligonucleotides to be employed as templates directing the synthesis of product nucleic acids, as explained further below, the sequences of the nucleic acids present on the array are selected based on the sequences of the desired product nucleic acids. Where desired, probe design algorithms, including but not limited to those algorithms described in U.S. Pat. No. 6,251,588 and published U.S. Application Nos. 20040101846; 20040101845; 20040086880; 20040009484; 20040002070; 20030162183 and 20030054346; may be employed.

Where nucleic acid arrays are employed as the starting materials, the number of nucleic acid features of the array may vary, where the number of features present on the surface of the array may be at least 1, 2, 5, or 10 or more, such as at least 20 and including at least 50, where the number may be as high as about 100, as about 500, as about 1000, as about 5000, as about 10000 or higher. In representative embodiments, the subject arrays have a density ranging from at least about 10 to about 100 to about 100,000 features/cm², such as from about 500 to about 20,000 features/cm², including from about 1000 to about 20,000 features/cm² in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. In certain representative embodiments, the density of the single-stranded nucleic acids may range from about 10⁻³ to about 1 pmol/mm², such as from about 10⁻² to about 0.1 pmol/mm², including from about 5×10⁻² to about 0.1 pmol/mm².

The length of the nucleic acids may vary considerably depending on the intended use of the purified nucleic acids to be produced in the final composition, and in representative embodiments ranges from about 10 to about 250 nucleotides, such as from about 15 to about 150 nucleotides, such as from 20 to 100 nucleotides and including from about 40 to about 80 nucleotides.

The nucleic acid sequences that are present on the surface of the array may be the same or different from one another, in that they may have the same or different sequences. In one representative embodiment, the nucleic acids all have at least one region with an identical nucleotide sequence. This common region may be any where from about 3 nucleotides to about 50 nucleotides, including about 10 nucleotides to about 25 nucleotides, such as from about 15 nucleotides to about 20 nucleotides in length, and may serve as a binding region, for instance, for a universal binding site or for a small nucleic acid binding agent (so as to facilitate separation in a physical separation binding step, as will be described in more detail below). In another representative embodiment, the nucleic acids generated in accordance with the methods herein described have both a common and a variable region, as will be described in greater detail below.

In practicing the subject methods, a silica containing solid support, such as an array, is contacted with a cleavage agent to produce fluid cleavage product that includes both silica and nucleic acids. In this step of the subject methods, the support is subjected to cleavage conditions sufficient to cleave or separate the surface immobilized nucleic acids from the surface of the silica containing substrate. In representative embodiments, this step may include contacting the array with an effective amount of a cleavage agent capable of cleaving the nucleic acids from surface of the substrate. Cleavage agents of interest include, but are not limited to: acid hydrolysis solutions (such as HF-based reagents), basic solutions, and the like.

In representative embodiments, the cleavage agent is a basic solution. Basic solutions of interest for use in the subject methods are any solutions that include a base and are sufficiently strong such that when contacted with the surface of the substrate, the desired fluid cleavage product that contains both solution phase nucleic acids and silica is produced. In representative embodiments, the basic solution employed as the cleavage agent is a solution having a pH from about 8 to about 14, such as from about 9 to about 13, and including from about 10 to about 12. In representative embodiments, the basic salt of the basic solution may be one having a pKₐ that ranges from about 8 to about 16, such as from about 9 to about 14, and including from about 10 to about 12. The concentration of the base in the solution may vary, but in representative embodiments ranges from about 1 M to about 9 M, such as from about 8 M to about 8.5 M. Representative solutions of interest as cleavage agents for use in the subject methods include, but are not limited to, solutions of ammonia,_methylamine, ethylamine and the like for basic solutions and Bu₄NF in THF, Pyridine/HF in THF, HF in Acetonitrile, SiF₄ in Acetonitrile, H₂SiF₆/TEA in acetonitrile and the like for acid hydrolysis cleavage, where in representative embodiments, the solution is an ammonia solution.

The chemical cleavage agent is contacted with the substrate for a period of time sufficient for the nucleic acids to be released from the surface of the support. In representative embodiments contact is maintained for a period of time ranging from about 0.5 h to about 144 h, such as from about 2 h to about 120 h, and including from about 4 h to about 72 h. Any convenient method may be used to contact the cleavage agent with the nucleic acid displaying substrate. For instance, contacting may include, but is not limited to: submerging, flooding, rinsing, spraying, etc. Contact may be carried out at any convenient temperature, where in representative embodiments contact is carried out at temperatures ranging from about 0° C. to about 60° C., including from about 20° C. to about 40° C., such as from about 20° C. to about 30° C.

In representative embodiments, the initial fluid cleavage product produced by the above cleavage step contains a population of nucleic acids that is substantially the same as the nucleic acids fabricated and present on the substrate surface before cleavage. By “substantially the same as” is meant that the amount of nucleic acids in the fluid cleavage product differs from the amount of nucleic acids present on the initial substrate by no more than about 400%, such as no more than about 200%. Furthermore, substantially all of the nucleic acids present on the substrate are present in the initial cleavage product. In representative embodiments, the initial fluid cleavage product contains an amount of silica. The amount of silica in the initial fluid cleavage product may vary, but in certain embodiments ranges from about 1 mg to about 10 mg, such as from about 1 mg to about 5 mg, including from about 1 mg to about 2 mg, e.g., for every 1 to 10 ml.

In a second step of the subject methods, the resultant fluid cleavage product is then purified to obtain a purified composition of solution phase nucleic acids. In the purified composition of solution phase nucleic acids produced in these embodiments, the amount of silica present in the final composition is substantially reduced as compared to the amount of silica present in the fluid cleavage product. By substantially reduced is meant that the amount of silica present is reduced by at least about 98%, such as by at least about 99%, and including by at least about 99.9%, e.g., (wt/wt).

In representative embodiments, the purification protocol may include a physical separation step in which the nucleic acids are separated from the silica in the composition by a physical separation protocol. Representative physical separation protocols include, but are not limited to: centrifugation, filtration, extraction, distillation, decanting, drying, and/or a combination thereof.

In representative embodiments, the purification is by centrifugation. Centrifugation may be carried out by any convenient protocol. For instance, a Spin Vac, a plate spinner, a microfuge, a small or large scale centrifuge, or the like, all of which are available for purchase from Eppendorf (Hamburg, Germany). In one embodiment, the initial cleavage product is placed into a sample container and the sample container is then placed in an appropriate centrifuge device and centrifuged for a sufficient amount of time so as to separate the solution phase nucleic acids from the silica, wherein a sufficient duration of time may range from about 3 minutes to about 30 minutes, including from about 5 minutes to about 20 minutes, such as from about 10 minutes to about 15 minutes. The speed of the centrifuge (i.e., revolutions per minute (rpms)) may vary depending on the type of centrifuge used, and in representative embodiments ranges between about 2,000 rpm to about 50,000 rpm, such as from about 5,000 rpm to about 30,000 rpm, and including from about 8,000 rpm to about 15,000 rpm.

In some embodiments it may be desirable to include one or more nucleic acid binding agents into the initial fluid cleavage product so as to facilitate the purification of the nucleic acids. A nucleic acid binding agent, as described above, may be any agent capable of facilitating isolation and/or purification of the solution phase nucleic acids, for instance, an oligonucleotide that binds to, e.g., via hybridization, the solution phase nucleic acids. In these embodiments where a common sequence is present in all of the nucleic acids present in the initial nucleic acid source, one may use a nucleic acid binding agent that hybridizes to the common sequence and therefore use a single type of nucleic acid binding agent. Alternatively, one may use a population of distinct or different nucleic acids as the nucleic acid binding agent, where the different members of the population hybridize to different domains of the nucleic acids that have been previously released from the substrate surface.

The above physical separation step serves to remove substantially all, if not all, of the particulate silica from the initial fluid cleavage product. A feature of the remaining fluid, referred to herein as the “intermediate fluid,” is that it is an aqueous solution of silica and nucleic acids, where the solution includes substantially little, if any, solid phase or particular silica. The remaining intermediate fluid which results from the physical separation step may then be subjected to one or more additional purification protocols, as desired.

In representative embodiments, the one or more additional purification protocols include a precipitation protocol. Any convenient precipitation protocol may be employed. In certain representative embodiments, the precipitation protocol is an alcohol precipitation protocol, such as an ethanol precipitation protocol, isopropanol precipation protocol, etc. Where the precipitation protocol employed is an alcohol precipitation protocol, in representative embodiments the monovalent cation concentration in the silica/nucleic acid aqueous fluid is first adjusted, as desired, e.g., by adding an appropriate one or more sources of monovalent cation, such as sodium acetate, sodium chloride, ammonium acetate, lithium chloride, etc., as is known in the art. Next, alcohol, e.g., ethanol, isopropanol, etc., is added to the aqueous fluid composition, where the amount of alcohol that is added may vary, but in representative embodiments is added in excess, such as in an amount that is at least 1 to 10 times or greater than the volume of the sample. Following appearance of the resultant nucleic acid precipitate, the nucleic acid precipitate may be recovered using any convenient protocol, e.g., by centrifugation. Following any desired rinse, e.g., with alcohol, any remaining alcohol may be removed by drying.

A feature of the above precipitation purification step is that it removes substantially all of the silica present in the intermediate fluid, where by substantially all is meant at least about 98%, such as at least about 99%, including at least about 99.9% of the silica present in the intermediate fluid, as determined using the OD methods described in the Experimental Section below. While removing substantially all of the silica present in the intermediate fluid, a significant amount of the nucleic acids remain in the fluid, such as at least about 25% or more, such as at least about 30% or more, including at least about 40% or more, as determined using the OD methods described in the Experimental Section below.

In representative embodiments of the subject invention, the purification may include one or more physical separation sub-steps that may be followed by one or more precipitation sub-steps. As many purification steps desired may be performed.

For instance, in a representative embodiment, a physical separation sub-step (e.g., centrifugation) is followed by two different precipitation sub-steps. The number of sub-steps may vary so long as the resultant product contains a reduced amount of silica. For instance, the resultant product should have a silica content that is substantially reduced as compared to the initial intermediate fluid product, and the initial fluid cleavage product. By substantially reduced is meant a final composition that contains about 50%, or about 60%, or about 75%, or about 80%, or about 90% less silica as compared to the initial cleavage product. As such, while the amount of silica in the final product may vary, in representative embodiments it will not exceed about 1%.

In representative embodiments, the final composition includes a substantial proportion of the nucleic acids that were present in the initial fluid cleavage product. By substantial proportion of nucleic acids is meant at least about 35%, such as at least about 40%, and including at least about 50%, e.g., at least about 75% or more of the nucleic acids present in the initial fluid cleavage product.

Accordingly, the above-described methods result in the production of a composition of substantially purified solution phase nucleic acids. By substantially purified is meant that the amount of silica in the resultant composition is reduced in comparison to the amount of silica present in the cleavage product. The purified nucleic acids may comprise a homogenous composition, in that all of the nucleic acids present in the final product have identical sequences, or the purified nucleic acids may comprise a heterogeneous composition, wherein at least one nucleic acid present in the composition differs from another nucleic acid present in the composition, depending on the intended use of the final composition. Where the final composition includes a heterogeneous mixture of nucleic acids, in representative embodiments the composition is characterized such that for each feature present on the initial array, there is at least one nucleic acid in the composition mixture that corresponds to the feature, where by corresponds is meant that the nucleic acid is one that is generated by cleavage of a surface immobilized nucleic acid of the feature of the initial chemical array. The length of each of the product nucleic acids present in the final composition ranges, in representative embodiments, from about 10 to about 250 nucleotides, such as from about 15 to about 150 nucleotides, including from about 20 to about 100 nucleotides, e.g., from about 40 to about 60 nt.

For those embodiments where the final composition of solution phase nucleic acids is a mixture, the term mixture refers to a heterogeneous composition of a plurality of different ribonucleic or deoxyribonucleic acids that differ from each other by residue sequence. Accordingly, the mixtures produced by the subject methods may be viewed as compositions of two or more nucleic acids that are not chemically combined with each other and are capable of being separated, e.g., by using an array of complementary surface immobilized nucleic acids, but are not in fact separated.

The number of different or distinct nucleic acids, i.e., of differing sequence, present in the final composition may vary, but is generally at least 2, including at least about 5, as well as at least about 10, such as at least about 20, at least about 50, at least about 100 or more, where the number may be as great as about 1000, about 5000 or about 25,000 or greater. Any two given nucleic acids in the final composition are considered distinct or different if they include a stretch of at least 20 nucleotides in length in which the sequence similarity is less then 98%, as determined using the FASTA program (using default settings).

In representative embodiments, the product nucleic acid composition may be characterized by having a known composition. By known composition is meant that, because of the way in which the composition is produced, the sequence of each distinct nucleic acid in the fluid cleavage product, and subsequently in the purification solution phase, can be predicted with a high degree of confidence. Thus, the sequence of each individual or distinct nucleic acid in the final composition is known. In many embodiments, the relative amount or copy number of each distinct nucleic acid of differing sequence in the final composition is known. Put another way, in certain embodiments the final composition of nucleic acids is known to include a constituent nucleic acid corresponding to each feature of the array used to produce it, such that each feature of the array is represented in the final composition.

The subject methods find use in a variety of different applications where a substrate bound nucleic acid is employed as an initial nucleic acid source in the production of a solution phase nucleic acid product. Of particular interest is the use of the subject methods in the array-based production of solution phase nucleic acid mixtures, where nucleic acid arrays are employed as initial sources the desired product nucleic acids, e.g., because nucleic acid arrays can be produced with known composition using in situ technology. Examples of such applications include applications where it is desired to produce a collection, e.g., library, of a plurality of different nucleic acids of known sequence.

While the subject methods can be used in a variety of different types of nucleic acid applications c, of interest are those applications described in U.S. patent application Ser. Nos. 09/628,472 and 11/008,603; where the applications disclosed in these applications may be readily modified to employ the composition of cleaved, solution phase purified nucleic acids produced by the subject methods. Specific representative applications of interest include, but are not limited to, using the product nucleic acids produced by the subject methods as: 1) template nucleic acids in primer extension reactions (to produce populations of gene specific primers); 2) template nucleic acids in strand displacement nucleic acid synthesis applications; 3) template nucleic acids in in vitro transcription reactions; and the like.

In yet other applications, the product nucleic acid compositions may be employed as modulatory agents, e.g., in gene silencing applications (where the nucleic acids are siRNA agents), etc. For example, having an initial source of nucleic acids that is an in situ produced a nucleic acid array of a plurality of distinct shRNA nucleic acids directed to the same or different targets, the methods may be employed to rapidly produce a library of shRNA molecules.

Kits

Also provided by the subject invention are kits for use in practicing the subject methods. Generally, the kits include a cleavage reagent for separating bound nucleic acids from a silica containing support (e.g., nucleic acid array) and a purification element. In one embodiment, the kit may contain a cleavage agent that is a basic solution. Additionally, in one embodiment, the kit may contain a purification element such as a physical separation element, a precipitation reagent, or both. In a further embodiment, a representative kit of the invention may also include a silica containing support to which is bound one or more nucleic acids.

A set of instructions may also be included, where the instructions may be associated with a package insert and/or the packaging of the kit or the components thereof. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another, means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

I. Nucleic Acid Array Preparation

DNA microarrays were manufactured according to the standard Agilent manufacturing process. Overall, an automated tool designed by Agilent Technologies was used in conjunction with standard phosphoramidite chemistry on a silylated 6.625×6 inch wafer using the following major modifications to produce surface immobilized nucleic acids of defined sequence. First, the solid support used was a flat, non-porous surface. Second, the coupling step was controlled in space using pulse-jet-printing technologies to deliver the appropriate amount of activator and phosphoramidite to the appropriate spatial location on the solid support. Third, the oxidation and detritylation reactions were performed in dedicated flowcells whose mechanical operations are described in the following paragraph. The oxidation solution was 0.02M I₂ in THF/Pyridine/H₂O and the detritylation solution was 3% DCA in Toluene.

Oxidation and detritylation reactions were carried out by flood steps in a flowcell as described below. The flowcell is constructed such that a glass substrate carrying the microarrays forms one wall of the reactor chamber. The substrate is brought to bear upon a seal embedded in the perimeter of the fixed reactor cell thus forming a high aspect ratio sealed chamber where, the aspect ratio is defined as the ratio of the planar flowcell width L to the lateral gap height h. Typically, the planar width of the substrate is 15-20 cm while the gap is 0.5 to 1 mm. Active liquid reagents, wash solvents and gases are introduced into the flowcell through two ports. One port is located at the bottom corner of the cell and one at the top. A series of solenoid valves control the inflow and outflow of reagents to these two ports. The flowcell is mounted such that the walls of the flowcell are vertical so that gravity assists draining. During a typical synthesis cycle, the reagents are first introduced in the flowcell from the bottom port until the flowcell is filled (fill time). The reagents are then left in the flowcell without mixing for 30 s for oxidation and 60 sec for detritylation. Finally, the reagents are drained from the bottom port (drain time), followed by washes using 2 flowcell volumes of ACN. This is typically 30 to 50 mL depending on the particular flowcell geometry.

II. Cleavage or Nucleic Acids from Array Surface

Deprotection and cleavage of the DNA from the surface was performed as described by Cleary et al.(Nature Methods (2004), 1 (3), 241-248) and the oligonucleotides recovered were lyophilized in Eppendorf tubes. Each array could contain up to 22,575 features, and the appropriate number of locations was left blanked when fewer oligonucleotides were desired.

III. Physical Separation

The initial cleavage product produced in Step II above was harvested and Spin Vac dried. 450 μl of dH₂O was added and the resultant solution was vortexed to provide for thorough mixing. The resultant fluid was then centrifuged at 8000 rpm for 10 minutes at room temperature. The supernatant was decanted to a new 1.5 ml microcentrifuge labeled as “sample” and 1.2 μl were taken for a UV absorption measurement by Nanodrop.

IV. Ethanol Precipitation

Next, the sample was purified, by adding 45 μl of 3 M sodium acetate and 1.0 ml of 100% ethanol to the tube and mixed well. The sample tube was then placed in a 4° C. refrigerator overnight. Afterward, the sample was spun for 30 min at 4° C. in a microcentrifuge at 14000 rpm and the supernatant was decanted using a P-200 Pipetman micropipettor and preserved in a 1.5 ml microcentrifuge tube labeled as “waste”. 200 μl of 70% ethanol was added to the sample tube and the resultant mixture was spun for 10 min at 4° C. in the microcentrifuge (14000 rpm). The supernatant was again decanted using a P-200 Pipetman micropipettor and preserved in the “waste” microcentrifuge tube. 42.5 μl of dH₂O, 7.5 μl of a random 25-mer (100 ng/μl), 5 μl of 3 M sodium acetate, and 110 μl of 100% ehanol were added, mixed well, and spun for min at 4° C. in the microcentrifuge (14000 rpm). Once again, the supernatant was decanted using a P-200 Pipetman micropipettor and preserved in the “waste” microcentrifuge tube. 200 μl of 70% ethanol was then added to the sample tube, spun for 30 min at 4° C. in the microcentrifuge (14000 rpm), and the supernatant was decanted using a P-200 Pipetman micropipettor and preserved in the “waste” microcentrifuge tube. The resultant sample was then centrifuged at 14000 rpm for 10 min at 4° C., a P-10 Pipetman micropipettor tip was then used to decant the liquid left over and the sample was then resuspended.

V. Determination of Nucleic Acid Purification

To evaluate the effectiveness of ethanol precipitation in separating nucleic acids from silica, the following protocol was performed. A 1 μl aliquot from the re-suspended solution was sampled and a UV absorbance measurement performed on a UV spectrophotometer (Nanodrop). After purification, another 1 μl aliquot from the purified solution was sampled and a UV absorbance measurement performed as previously. The efficiency of the purification was determined from the comparison of the two UV measurement.

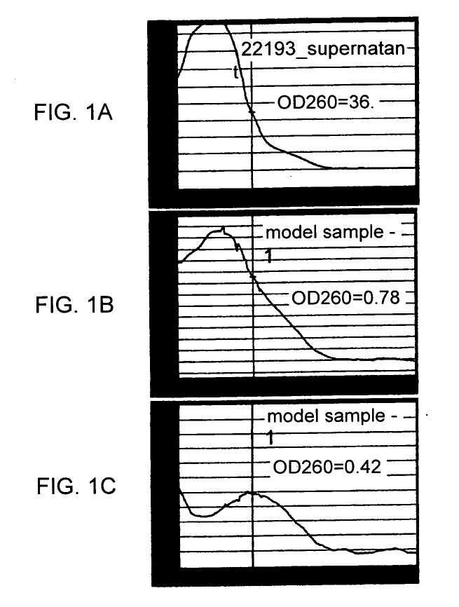

As can be seen by FIG. 1A, the initial cleavage product had a high concentration of silica, as indicated by the strong absorption between 230 nm to 270 nm (OD260=36). After the first ethanol precipitation, a large amount of silica compounds were removed (OD 260=0.78). See FIG. 1B. After the second ethanol precipitation, the amount of silica was substantially reduced (OD 260=0.42). However, about 40% of the amount of the initial nucleic acids were retained in the sample. See FIG. 1C.

The above results demonstrate that ethanol precipitation is an effective way to separate silica from nucleic acids in a fluid mixtures of silica and solution phase nucleic acids.

Although the foregoing invention has been described in some detail by way of illustration and example for purposes of clarity of understanding, it is readily apparent to those of ordinary skill in the art in light of the teachings of this invention that certain changes and modifications may be made thereto without departing from the scope of the appended claims.

1. A method of separating a nucleic acid from a surface of a silica comprising support to which said nucleic acid is bonded, said method comprising:
(a) contacting said surface with a cleavage agent for a duration of time sufficient to separate said nucleic acid from said surface and produce a fluid cleavage product comprising said separated nucleic acid and silica; and
(b) subjecting said fluid cleavage product to a physical separation protocol to produce a fluid intermediate product containing said nucleic acid: and
(c) subjecting said fluid intermediate product to a precipitation protocol to produce a final composition, wherein the amount of silica present in said final composition is substantially reduced as compared to the amount of silica present in said fluid cleavage product.

2. The method according to claim 1, wherein said nucleic acid is DNA.

3. The method according to claim 1, wherein said nucleic acid is RNA.

4. The method according to claim 1, wherein said nucleic acid is up to about 150 nucleotides in length.

5. The method according to claim 1, wherein said nucleic acid is up to about 100 nucleotides in length.

6. The method according to claim 1, wherein said silica comprising support of bonded nucleic acids comprises an array of nucleic acids.

7. The method according to claim 1, wherein said array of nucleic acids comprises at least about 100 different features of nucleic acids.

8. The method according to claim 7, wherein said features are present at a density of at least about 10 features/cm².

9. The method according to claim 1, wherein said silica comprising support is glass.

10. The method according to claim 1, wherein said cleavage agent comprises a base.

11. The method according to claim 10, wherein said base comprises ammonia.

12. The method according to claim 1, wherein said duration of time is from about 0.5 hr to about 144 hrs.

13. (canceled)

14. The method according to claim 1, wherein said physical separation step comprises a method selected from the group consisting of: centrifugation, filtration, extraction, distillation, decanting, and drying.

15. The method according to claim 14, wherein said physical separation step is centrifugation.

16. (canceled)

17. The method according to claim 1, wherein said precipitation is an alcohol precipitation.

18. The method according to claim 17, wherein said alcohol precipitation is ethanol precipitation.

19. The method according to claim 1, wherein said physical separation protocol and said precipitation protocol includes two different sub-steps.

20. The method according to claim 19, wherein said two different sub-steps comprise centrifugation.

21. The method according to claim 20, further comprising ethanol precipitation following said centrifugation.

22. The method according to claim 21, further comprising a second ethanol precipitation sub-step.

23. The method according to claim 1, wherein said method produces a final composition that has at least about 75% less silica as compared to said fluid cleavage product.

24. The method according to claim 1, wherein said final composition comprises at least about 40% of the nucleic acids present in said initial fluid cleavage product.

25. The method according to claim 1, further comprising adding a nucleic acid binding agent to said fluid cleavage product prior to said purifying.

26. A nucleic acid composition produced by the method according to claim 1.

27. A kit comprising:
(a) a cleavage agent for separating said nucleic acids from a silica comprising support; and
(b) a purification element.

28. The kit according to claim 27, wherein said cleavage agent is a base.

29. The kit according to claim 28, wherein said base is ammonia.

30. The kit according to claim 27, wherein said purification element comprises a precipitation reagent.

31. The kit according to claim 27, wherein said kit further comprises a silica comprising support comprising a nucleic acid bonded to the surface thereof.

32. A method of separating nucleic acids from silica in a fluid comprising silica and nucleic acids, said method comprising:
contacting said fluid with an amount of alcohol effective to cause nucleic acids present in said fluid to precipitate, such that a precipitate of nucleic acids in said fluid is produced; and
separating said precipitate from said fluid to separate nucleic acids from silica in said fluid.

33. The method according to claim 32, wherein said alcohol is ethanol.
